(19) United States
(12) Patent Application Publication
(10) Pub. No.: US 2017/0079822 A1
(43) Pub. Date: Mar. 23, 2017

(54) GASTRIC RECYCLING APPARATUS AND METHODS FOR OBESITY TREATMENT

(71) Applicant: Mayo Foundation for Medical Education and Research, Rochester, MN (US)

(72) Inventors: Gianrico Farrugia, Rochester, MN (US); Paul A. Friedman, Rochester, MN (US); Elizabeth Rajan, Rochester, MN (US); Charles J. Bruce, South Ponte Vedra Beach, FL (US); Samuel J. Asirvatham, Rochester, MN (US); Michael J. Levy, Rochester, MN (US); Louis-Michel Wong Kee Song, Rochester, MN (US); Juliane Bingener-Casey, Rochester, MN (US); Navtej S. Buttar, Rochester, MN (US)

(21) Appl. No.: 15/126,620

(22) PCT Filed: Mar. 19, 2015
(86) PCT No.: PCT/US2015/021397

§ 371 Date: Sep. 16, 2016

Related U.S. Application Data
(60) Provisional application No. 61/955,858, filed on Mar. 20, 2014.

(51) Int. Cl. A61F 5/00

(52) U.S. Cl. CPC A61F 5/0076; A61F 5/0013; A61B 17/1114

(57) ABSTRACT

Obesity treatment apparatus and methods are described herein involving gastric recycling of food are described herein. In one or more embodiments, the gastric recycling apparatus keeps returning chyme to the stomach increasing satiety signals and delaying hunger signals associated with an empty stomach. It may also may slow the emptying of the stomach as the caloric load in the stomach delays emptying and/or limit absorption of food within the small intestine by delivering at least a portion of food passing through the small intestine back into the stomach.

### RELATED APPLICATION

The present application claims the benefit under 35 U.S.C. §119(e) of U.S. Provisional Patent Application No. 61/955,858, filed on Mar. 20, 2014 and titled GASTRIC RECYCLING APPARATUS AND METHODS FOR OBESITY TREATMENT, which is hereby incorporated by reference in its entirety.

Obesity treatment apparatus and methods involving gastric recycling of food are described herein.

Morbid obesity and its concurrent health risks (including diabetes, heart disease and other ailments) are of near-epidemic proportions in industrialized societies. A wide variety of treatments have been proposed to attempt to treat morbid obesity. These have a wide variety of efficacy and associated morbidity. These treatments include techniques to reduce stomach volume, alter gastric and intestinal motility, and alter the absorption of nutrients in the small intestine.

Clearly, obesity is a complex disease having physiologic, social and psychological components which are not fully understood. The complex nature and the enormous societal implication of obesity require a wide variety of treatment options be available to permit a physician to select a most appropriate option for a particular patient.

Even if all treatments were proven effective, no one treatment can meet the clinical needs presented by a diverse population. For example, bariatric surgeries, such as the Roux-en-Y procedure as will be described, is not considered suitable for so-called mildly obese patients (e.g., those with a Body Mass Index less than 35) given the cost, adverse event profile, and irreversible alteration of the anatomy.

Less invasive procedures (such as gastric banding) have reduced surgical risk. Unfortunately, they suffer from reduced efficacy (and they are not without risks). Further, efficacy may be culturally biased. Namely, gastric banding studies show reduced efficacy in North American patients compared to European patients.

Surgical approaches may include, e.g., minimally invasive surgery, open surgery and endoscopic approaches to gastric volume reduction. Gastric volume reduction procedures include vertical and horizontal gastroplasty in which sutures, staples or other fixation devices are used to join opposing surfaces of the stomach to create a reduced volume pouch and thereby reduce caloric intake. Less invasive techniques for restricting the volume of the stomach also include a gastric partition in which the stomach wall is endoscopically cinched together to form a reduced size pouch. Other techniques for reducing gastric volume size include placement of obstructions within the stomach. Gastric volume reduction effectiveness tends to markedly decrease over time, likely from adaptation.

To address deficiencies associated with gastric volume reduction, treatments have been suggested and developed for reducing the amount of nutrient absorption in the small intestine (particularly in the upper and middle portions of the small intestine—the duodenum and jejunum, respectively). Techniques to reduce nutrient absorption (commonly referred to as malabsorption treatments) include drug therapies for reducing lipid absorption. However, these interventions are associated with unacceptable side effects.

Other malabsorption treatments include surgical techniques for rerouting the intestinal system to bypass an extended portion of the small intestine. These include a so-called jejunoileal bypass. Not commonly used due to unacceptable mortality rates, a jejunoileal bypass typically results in effective weight loss. Other techniques include the gastric bypass (or Roux-en Y) and duodenal switch. As a result, the absorptive length of the small intestine is significantly shortened thereby reducing the amount of nutrients which are absorbed into the body and which support or lead to weight gain. These procedures combine the benefits of gastric volume reduction with malabsorption.

Less invasive techniques are suggested for placing a band (referred to as LAP bands) around an upper portion of the stomach to act as a belt to reduce the size of the stomach and create a small passageway (a stoma) from a small upper pouch to the remainder of the stomach or a sleeve that covers the upper small intestine. Feelings of less satiation and subsequent behavior to achieve satiation often limit effectiveness of the above therapies.

### SUMMARY

Obesity treatment apparatus and methods are described herein involving gastric recycling of food are described herein. In one or more embodiments, the gastric recycling apparatus keeps returning chyme from the small bowel to the stomach increasing satiety signals and delaying hunger signals associated with an empty stomach. It may also slow the emptying of the stomach as the caloric load in the stomach delays emptying and/or limit absorption of food within the small intestine by delivering at least a portion of food passing through the small intestine back into the stomach.

In a first aspect, one or more embodiments of the apparatus configured to promote gastric recycling as described herein may include: a passageway comprising a passageway inlet and a passageway outlet, wherein a primary flow axis extends through the passageway from the passageway inlet to the passageway outlet; a gastric outlet branch in fluid communication with the passageway at a location between the passageway inlet and the passageway outlet, the gastric outlet branch comprising a gastric outlet configured for placement in a stomach when the passageway is located in a small intestine, wherein the gastric outlet branch defines a gastric outlet flow axis extending through the gastric outlet branch from the passageway to the gastric outlet, and wherein material entering the passageway through the inlet exits the passageway through either the gastric outlet branch or the passageway outlet; a one-way valve configured to allow movement of material out of the passageway and into the gastric outlet branch and to restrict movement of material from the gastric outlet branch into the passageway; and gastric fixation apparatus positioned on an exterior of the gastric outlet branch, wherein the gastric fixation apparatus comprises a delivery configuration and an expanded configuration, wherein the gastric fixation apparatus is larger in a radial direction extending outwardly from the gastric outlet flow axis when the gastric fixation apparatus is in the expanded configuration than in the delivery configuration such that movement of the gastric outlet branch through an opening surrounding the exterior of the gastric outlet branch is restricted in at least one direction by the gastric fixation apparatus in the expanded configuration.

In one or more embodiments of the first aspect, the passageway inlet is positioned on an inlet branch located upstream from the location at which the gastric outlet branch is in fluid communication with the passageway, and wherein the inlet branch is configured to position the inlet in the small intestine when the passageway and the passageway outlet are located in the small intestine. In one or more embodiments, the apparatus further comprises an inlet branch fixation apparatus positioned on an exterior of the inlet branch, wherein the inlet branch fixation apparatus comprises a delivery configuration and an expanded configuration, wherein the inlet branch fixation apparatus is larger in a radial direction extending outwardly from the primary flow axis when the inlet branch fixation apparatus is in the expanded configuration than in the delivery configuration such that movement of the inlet branch within the small intestine is restricted when the inlet branch fixation apparatus is in the expanded configuration.

In one or more embodiments of the first aspect, the passageway inlet is positioned on an inlet branch located upstream from the location at which the gastric outlet branch is in fluid communication with the passageway, wherein the inlet branch is configured position the passageway inlet at or upstream of the pyloric valve when the passageway and the passageway outlet are located in the small intestine, and wherein the inlet branch comprises a sleeve extending through the small intestine to the location at which the gastric outlet branch is in fluid communication with the passageway. In one or more embodiments, the apparatus further comprises an inlet branch fixation apparatus positioned on an exterior of the inlet branch, wherein the inlet branch fixation apparatus comprises a delivery configuration and an expanded configuration, wherein the inlet branch fixation apparatus is larger in a radial direction extending outwardly from the primary flow axis when in the expanded configuration than in the delivery configuration such that movement of the inlet branch downstream from the pyloric valve is restricted when the inlet branch fixation apparatus is in the expanded configuration.

In one or more embodiments of the first aspect, the apparatus comprises a pass-through passage configured to provide a passage through a body lumen in which the passageway is located such that material passing through the body lumen can move from a location upstream of the passageway inlet to a location downstream of the passageway outlet outside of the passageway. In one or more embodiments, the pass-through passage comprises a pass-through inlet, and wherein one or both of the passageway inlet and the pass-through inlet are configured to change size within the lumen such that varying amounts of material passing through the body lumen enter the passageway through the passageway inlet or the pass-through passage through the pass-through inlet.

In one or more embodiments of the first aspect, the passageway outlet is located in an outlet branch extending away from the location at which the gastric outlet branch is in fluid communication with the passageway such that the passageway outlet is spaced apart from a junction of the outlet branch and the gastric outlet branch. In one or more embodiments, an outlet branch fixation apparatus is positioned on an exterior of the outlet branch, wherein the gastric fixation apparatus comprises a delivery configuration and an expanded configuration, wherein the outlet branch fixation apparatus is larger in a radial direction extending outwardly from the primary flow axis when the outlet branch fixation apparatus is in the expanded configuration than in the delivery configuration such that movement of the outlet branch within a body lumen in which the outlet branch is located is restricted when the outlet branch fixation apparatus is in the expanded configuration. In one or more embodiments, the passageway outlet comprises a variable flow restrictor configured to provide an opening configured to be selectively enlarged or reduced in size to change the flow restriction provided by the variable flow restrictor. In one or more embodiments, the variable flow restrictor comprises a mechanical iris comprising a plurality of movable leaves that are configured to move to change the size of the opening in the variable flow restrictor. In one or more embodiments, the variable flow restrictor comprises an expandable bladder configured to be inflated or deflated to change the size of the opening in the variable flow restrictor.

In one or more embodiments of the first aspect, the passageway outlet comprises a plurality of openings in a wall of the passageway.

In a second aspect, one or more embodiments of an apparatus configured to promote gastric recycling as described herein may include: a passageway comprising a passageway inlet and a passageway outlet, wherein a primary flow axis extends through the passageway from the passageway inlet to the passageway outlet, wherein the passageway comprises a gastric portion and a small intestine portion, wherein the gastric portion is located between the passageway inlet and the small intestine portion and wherein the small intestine portion is located between the gastric portion and the passageway outlet; fixation apparatus positioned on an exterior of the passageway, wherein the fixation apparatus is configured to fix the position of the passageway within a fistula formed between a stomach and a small intestine such that the gastric portion is located within a stomach and the small intestine portion is located within a small intestine; and one or more openings formed in a wall forming the gastric portion of the passageway, wherein the one or more openings are configured to allow material entering the passageway inlet of the passageway in the small intestine to flow out of the one or more openings, and wherein material moving through the gastric portion of the passageway that does not flow out of the one or more openings moves through the small intestine portion of the passageway to the passageway outlet.

In one or more embodiments of the second aspect, the passageway inlet is configured to allow a portion of material moving through a small intestine to pass the inlet without entering the passageway.

In a third aspect, one or more embodiments of a method of obesity control as described herein may include: capturing at least a portion of food passing through a small intestine in a passageway inlet of a passageway located in the small intestine; delivering at least a portion of food in the passageway into the stomach; preventing food from passing directly into the passageway from the stomach; and allowing at least a portion of the food passing through the small intestine to pass through the small intestine downstream of the passageway.

In one or more embodiments of the third aspect, delivering at least a portion of food in the passageway into the stomach comprises moving the food out of the passageway through a gastric outlet branch comprising a gastric outlet located in the stomach. In one or more embodiments, a one-way valve located in the gastric outlet branch prevents food from passing directly into the passageway from the stomach.

In one or more embodiments of the third aspect, allowing at least a portion of the food passing through the small intestine to pass through the small intestine downstream of the passageway comprises passing the food out of the passageway through a passageway outlet located downstream of the passageway inlet.

In one or more embodiments of the third aspect, allowing at least a portion of the food passing through the small intestine to pass through the small intestine downstream of the passageway comprises allowing the food to bypass the passageway inlet such that the food does not enter the passageway.

As used herein and in the appended claims, the singular forms “a,” “an,” and “the” include plural referents unless the context clearly dictates otherwise. Thus, for example, reference to “a” or “the” component may include one or more of the components and equivalents thereof known to those skilled in the art. Further, the term “and/or” means one or all of the listed elements or a combination of any two or more of the listed elements.

It is noted that the term “comprises” and variations thereof do not have a limiting meaning where these terms appear in the accompanying description. Moreover, “a,” “an,” “the,” “at least one,” and “one or more” are used interchangeably herein.

The above summary is not intended to describe each embodiment or every implementation of the obesity treatment apparatus or methods described herein. Rather, a more complete understanding of the invention will become apparent and appreciated by reference to the following Description of Illustrative Embodiments and claims in view of the accompanying figures of the drawing.

### BRIEF DESCRIPTION OF THE VIEWS OF THE DRAWING

FIG. 1 depicts one illustrative embodiment of a gastric recycling apparatus as described herein in location within the small intestine and stomach.

FIG. 2 depicts one illustrative embodiment of a variable flow restrictor that may be used in connection with the gastric recycling apparatus described herein.

FIGS. 3A & 3B depict another illustrative embodiment of a variable flow restrictor that may be used in connection with the gastric recycling apparatus described herein.

FIG. 4 depicts another illustrative embodiment of a gastric recycling apparatus as described herein in location within the small intestine and stomach.

FIG. 5 depicts another illustrative embodiment of a gastric recycling apparatus as described herein in location within a small intestine and stomach.

FIG. 6 depicts another illustrative embodiment of a gastric recycling apparatus as described herein in location within a small intestine and stomach.

FIG. 7 depicts another illustrative embodiment of a gastric recycling apparatus as described herein in location within a small intestine and stomach.

FIG. 8 depicts one illustrative embodiment of a port that may be used to inflate and/or deflate the fixation apparatus that may be used in connection with the gastric recycling apparatus described herein.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments. It is to be understood that other embodiments may be utilized and structural changes may be made without departing from the scope of the present invention.

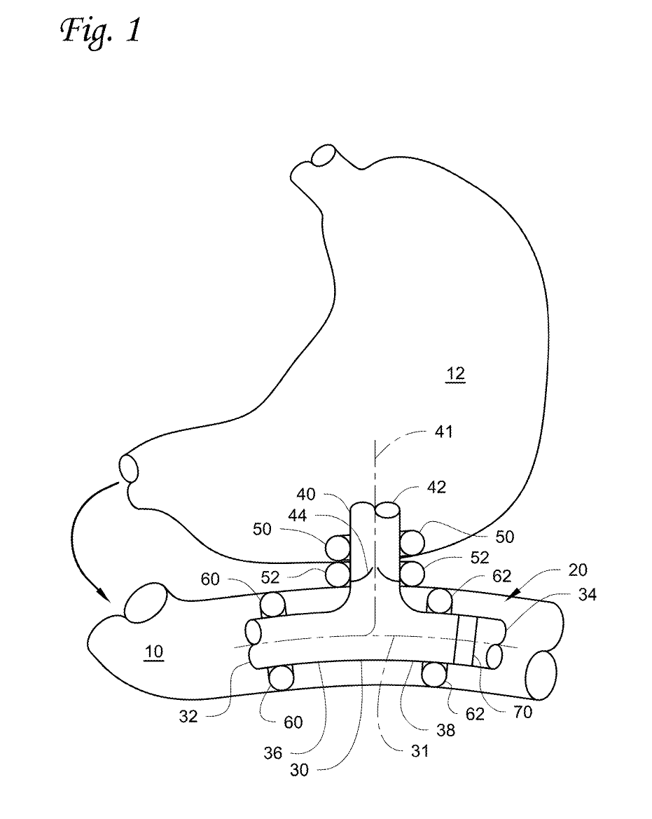

One illustrative embodiment of an apparatus as described herein is depicted in FIG. 1 in location within a patient. The apparatus can be described, in one or more embodiments, as being located within the small intestine in the second part of the duodenum and extending up to the ligament of Trietz. In one or more alternative embodiments, the apparatus can be described as being located within the small intestine in the second part of the duodenum and extending to the third part of the duodenum. In one or more alternative embodiments, the apparatus can be described as being located within the small intestine in the second part of the duodenum and may extend into the jejunum and ileum.

The apparatus 20 includes a passageway 30 comprising a passageway inlet 32 and a passageway outlet 34. A primary flow axis 31 extends through the passageway 30 from the passageway inlet 32 to the passageway outlet 34. In use, at least a portion of food passing through the small intestine 10 from the stomach 12 enters the passageway 30 through the passageway inlet 32 and passes through the passageway 30 to the passageway outlet 34.

The apparatus 30 also includes a gastric outlet branch 40 in fluid communication with the passageway 30 at a location between the passageway inlet 32 and the passageway outlet 34 of the passageway 30. The gastric outlet branch 40 includes gastric outlet 42 positioned in the stomach 12 of the patient when the passageway 30 is located in a small intestine 10. In one or more embodiments, the gastric outlet branch 40 defines a gastric outlet flow axis 41 extending through the gastric outlet branch 40 from the passageway 30 to the gastric outlet 42.

In one or more embodiments, at least a portion of food entering the passageway 30 through the passageway inlet 32 exits the passageway 30 through the gastric outlet 42 of the gastric outlet branch 40 so that it re-enters the stomach 12 rather than passing distally from the passageway outlet 34 of the passageway 30. In one or more embodiments of the apparatus described herein, food entering the passageway 30 through the passageway inlet 32 leaves the passageway 30 through either the gastric outlet branch 40 or the passageway outlet 34.

In one or more embodiments, the apparatus 20 includes a one-way valve 44 configured to allow movement of food out of the passageway 30 into the gastric outlet branch 40 so that it can enter the stomach 12 through the gastric outlet 42. The one-way valve 44 also restricts movement of food from the stomach 12 into the passageway 30 through the gastric outlet branch 40. In other words, the one-way valve 44 prevents food in the stomach 12 from passing out of the stomach 12 through the gastric outlet branch 40.

The one-way valve 44 may be provided in any suitable construction. Examples of some potentially suitable constructions for a one-way valve used in the apparatus 20 described herein may include, e.g., duckbill valves, flapper valves, ball valves, sock type valves or remotely switchable valve (e.g. electronic, magnetic) to potentially modify the function or even disable the valve based on clinical response, etc. In one or more embodiments, the apparatus 20 may include gastric fixation apparatus 50 positioned on an exterior of the gastric outlet branch 40. In one or more embodiments, the gastric fixation apparatus 50 may include a delivery configuration and an expanded configuration. The gastric fixation apparatus 50 may, in one or more embodiments, be larger in a radial direction extending outwardly from a gastric outlet flow axis 41 when the gastric fixation apparatus 50 is in the expanded configuration than in the delivery configuration such that movement of the gastric outlet branch 40 through an opening surrounding the exterior of the gastric outlet branch 40 is restricted in at least one direction by the gastric fixation apparatus 50 in the expanded configuration.

In particular, as seen in FIG. 1, the gastric fixation apparatus 50, which is depicted in its expanded configuration would prevent movement of the gastric outlet branch 40 out of the stomach 12 through the opening formed to allow insertion of the gastric outlet branch 40 into the stomach 12. In the delivery configuration, the gastric fixation apparatus 50 may have a smaller radial dimension such that it can be inserted through an opening formed in the stomach wall and the small intestine before taking on a larger radial dimension which helps to fix the location of the gastric outlet branch 40 with respect to both the small intestine 10 and the stomach 12. In one or more embodiments, the gastric fixation apparatus 50 may be in the form of an inflatable balloon or other expandable structure such as, e.g., expandable struts or other mechanical structures (such as, e.g., self-expandable metal stents or other structures that are advanced into place while “captured”—e.g., constrained within a sheath or other constraint that, upon removal, allows the device to assume a configuration that fixes the apparatus in place).

In one or more embodiments, the gastric station apparatus 50 may be supplemented by a secondary gastric fixation apparatus 52. In particular, the gastric fixation apparatus 50 may be positioned on the gastric outlet 40 such that the gastric fixation apparatus 50 is located within the stomach 12. In contrast, the secondary gastric fixation apparatus 52 may be positioned on the gastric outlet 40 such that it is positioned outside of the stomach 12 such that a portion of the stomach wall is located between the inner gastric fixation apparatus 50 and the outer gastric fixation apparatus 52. In one or more embodiments, the secondary or outer gastric fixation apparatus 52 may be located outside of the small intestine 10 as seen in, e.g., FIG. 1, such that the secondary or outer gastric fixation apparatus 52 is located between the stomach 12 and the small intestine 10. In one or more alternative embodiments, the secondary gastric fixation apparatus 52 may potentially be located within the small intestine 10.

In the delivery configuration, the secondary gastric fixation apparatus 52 may have, in one or more embodiments, a smaller radial dimension such that it can be inserted through an opening formed in the small intestine before taking on a larger radial dimension which helps to fix the location of the gastric outlet branch 40 with respect to both the small intestine 10 and the stomach 12. Furthermore, the secondary gastric fixation apparatus 52 may be constructed similarly to the gastric fixation apparatus 50 as described herein.

In one or more embodiments, the passageway inlet 32 into the passageway 30 is positioned on an inlet branch 36 of the passageway 30 that is located upstream from the location at which the gastric outlet branch 40 is attached to and/or in fluid communication with the passageway 30. The inlet branch 36, in such an embodiment, may be configured to position the passageway inlet 32 into the passageway 30 in the small intestine 10 when the passageway 30 and the pass-through outlet are also both located in the small intestine 10.

In one or more embodiments, the apparatus described herein may include an inlet branch fixation apparatus 60 positioned on an exterior of the inlet branch 36. The inlet branch fixation apparatus 60 may have a delivery configuration and an expanded configuration. In particular, the inlet branch fixation apparatus 60 is larger in a radial direction extending outwardly from the primary flow axis 31 when the inlet branch fixation apparatus 60 is in the expanded configuration than in the delivery configuration. As a result, movement of the inlet branch 36 within the small intestine 10 is restricted when the inlet branch fixation apparatus 60 is in the expanded configuration. Although movement of the inlet branch 36 within the small intestine 10 is described as being restricted, some movement of the inlet branch 36 within the small intestine 10 may be allowable without significantly degrading performance of the apparatus 20.

In the delivery configuration, the inlet branch fixation apparatus 60 may have, in one or more embodiments, a smaller radial dimension such that it can be advanced into the small intestine before taking on a larger radial dimension which helps to restrict movement of the inlet branch 36 within the small intestine 10. Furthermore, the inlet branch fixation apparatus 60 may be constructed similarly to the gastric fixation apparatus 50 as described herein.

In one or more embodiments of the apparatus described herein, the passageway outlet 34 of the passageway 30 is located in an outlet branch 38 that extends away from the location at which the gastric outlet branch 40 is in fluid communication with the passageway 30 such that the passageway outlet 34 is spaced apart from a junction of the outlet branch 38 and the gastric outlet branch 40.

In one or more embodiments in which the apparatus 20 includes an outlet branch 38, the apparatus 20 may also include an outlet branch fixation apparatus 62 positioned on an exterior of the outlet branch 38. The gastric fixation apparatus 62 may have a delivery configuration and an expanded configuration, wherein the outlet branch fixation apparatus the two is larger in a radial direction extending outwardly from the primary flow axis 31 when the outlet branch fixation apparatus 62 is in the expanded configuration than in the delivery configuration. As a result, movement of the outlet branch 38 within a body lumen such as, e.g., a small intestine 10, in which the outlet branch 38 is located is restricted when the outlet branch fixation apparatus 62 is in the expanded configuration. Although movement of the outlet branch 38 within the small intestine 10 is described as being restricted, some movement of the outlet branch 38 within the small intestine 10 may be allowable without significantly degrading performance of the apparatus 20.

In the delivery configuration, the outlet branch fixation apparatus 62 may have, in one or more embodiments, a smaller radial dimension such that it can be advanced into the small intestine before taking on a larger radial dimension which helps to restrict movement of the outlet branch 38 within the small intestine 10. Furthermore, the outlet branch fixation apparatus 62 may be constructed similarly to the gastric fixation apparatus 50 as described herein.

In one or more embodiments, the apparatus 20 may include a variable flow restrictor 70 positioned in the passageway 30 between the location at which the gastric outlet branch 40 is in fluid indication with the passageway 30 and the passageway outlet 34. In one or more embodiments, the variable flow restrictor 70 may be positioned in the passageway outlet 34. The variable flow restrictor 70 is configured to provide an opening that can be selectively enlarged or reduced in size to change the flow restriction provided by the variable flow restrictor 70.

Changing the flow restriction provided by the variable flow restrictor 70 may, in one or more embodiments, caused a corresponding increase or decrease in the amount of food passing through the apparatus 20 which reenters the stomach 12 through the gastric outlet branch 40. In particular, reducing the size of an opening in the variable flow restrictor 70 may increase the amount of food diverted into the stomach 12 through the gastric outlet branch 40, while increasing the size of the opening in the variable flow restrictor may decrease the amount of food diverted into the stomach 12 through the gastric outlet branch 40.

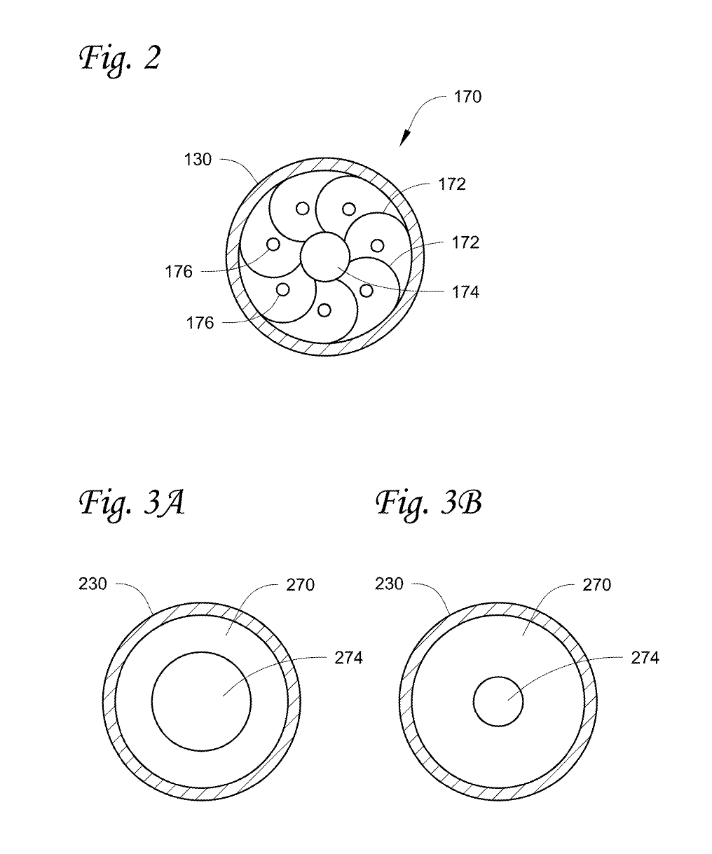

One illustrative embodiment of a variable flow restrictor 170 that may be used in connection with the apparatus described herein is depicted in FIG. 2 in a view taken along the primary flow axis 31 through the passageway 30 of the apparatus depicted in FIG. 1. The variable flow restrictor 170 positioned in the passageway 130 is in the form of a mechanical iris that includes a plurality of movable leaves 172 that are configured to move to change the size of the opening 174 in the variable flow restrictor 170. In many respects, the mechanical iris may be constructed according to the principles of a camera shutter or similar device. In one or more embodiments, one or more leaves 172 of the mechanical iris 170 may include one or more secondary openings 176 through which food could pass through the flow restrictor 170 even if the central opening 174 were completely closed.

Another illustrative embodiment of a variable flow restrictor 270 positioned in a passageway 230 that may be used in connection with the apparatus described herein is depicted in FIGS. 3A and 3B. The variable flow restrictor 270 is in the form of an expandable bladder that can be inflated or deflated to change the size of the opening 274 in the variable flow restrictor 270. In particular, the variable flow restrictor 270 is depicted with a larger central opening 274 in FIG. 3A. Expansion of the inflatable bladders in the flow restrictor 270 reduces the size of the opening 274 as depicted in, e.g., FIG. 3B.

Although not depicted in the figures, in one or more embodiments of the apparatus described herein a variable flow restrictor may be located within the gastric outlet branch 42 control the amount of fluid allowed to pass through the gastric outlet branch 40 into the stomach 12. Such a variable flow restrictor would be typically provided in addition to a one-way valve and, in one or more embodiments, the functions of the one-way valve and the variable flow restrictor may be incorporated into the same device positioned in the gastric outlet branch 40.

In one or more embodiments of the apparatus described herein, some control over the passage of the passage of food through the small intestine may be provided as the food leaves the stomach. One illustrative embodiment of such an apparatus is depicted in FIG.

4, where the apparatus 320 is positioned in the small intestine 310. Like the apparatus described elsewhere herein, the apparatus 320 includes a gastric outlet branch 340 in communication with a passageway 330. The passageway 330 includes a passageway outlet 334 which may include an optional fixation apparatus 362 as described herein. Similarly, the gastric outlet branch may include fixation apparatus 350 located in the stomach 312 and secondary fixation apparatus 352 located outside of the stomach 312 as described herein in connection with, e.g., apparatus 20 as depicted in FIG. 1. In addition, the apparatus 320 includes small intestine fixation apparatus 354 which may be positioned closer to the small intestine 310 such that the secondary fixation apparatus 352 is located between the small intestine fixation apparatus 354 and the wall 313 of the stomach 312. Such an arrangement of fixation apparatus 350, 352, and 354 may be useful in situations where, e.g., the distance between the small intestine 310 and the wall 313 of the stomach 312 is larger.

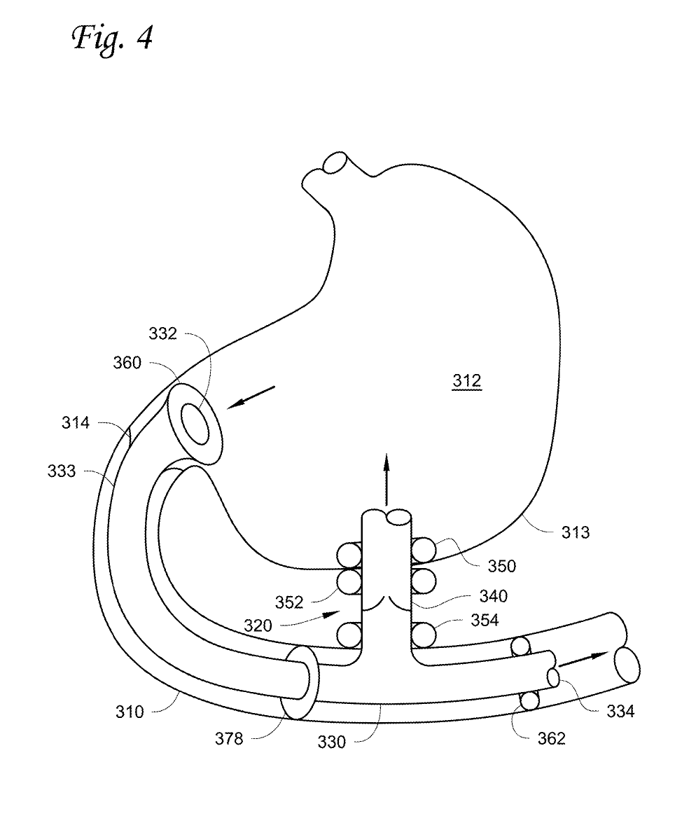

Another optional feature of the apparatus described herein depicted in connection with the illustrative embodiment of FIG. 4 is a flow restrictor 378 which may be positioned upstream of the location at which the gastric outlet branch 340 is in fluid communication with the passageway 330. The flow restrictor 378 may, in one or more embodiments, be in the form of a variable flow restrictor as described herein and may be used to slow the entry of fluid into the passageway 330. In one or more embodiments, a flow restrictor (not shown in FIG. 4) may be positioned in the gastric outlet branch 340 to restrict flow through the gastric outlet branch 340 into the stomach 312. Still another optional feature of the apparatus described herein that is depicted in connection with the illustrative embodiment of FIG. 4 is a sleeve 333 that may be used to effectively move the passageway inlet 332 to a position at or proximal of the pyloric valve 314. As a result, food would enter the apparatus 320 and pass through sleeve 333 without contacting the small intestine between the passageway inlet 332 and the passageway outlet 334. In such an embodiment, the apparatus 320 may include fixation apparatus 360 that is positioned at or proximal to the pyloric valve to maintain the position of the passageway inlet 332 at or proximal to the pyloric valve.

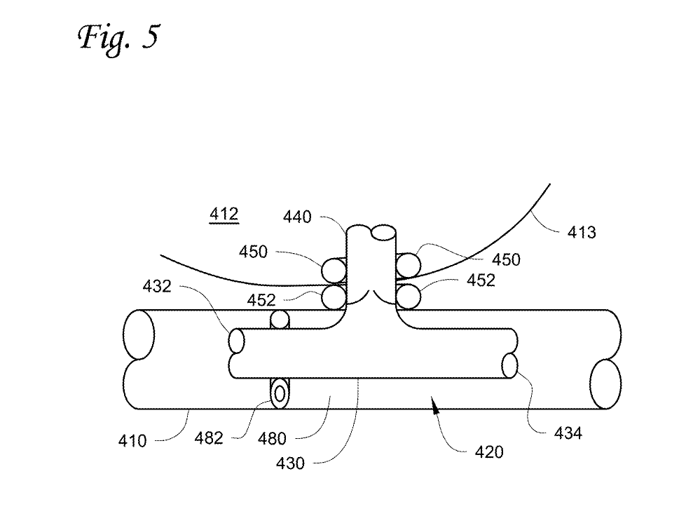

Another illustrative embodiment of an apparatus as described herein is depicted in FIG. 5. The apparatus 420 includes a passageway 430 and a gastric outlet branch 440 as described herein in connection with the other illustrative embodiments. The gastric outlet branch includes fixation apparatus 450 and 452 (on opposite sides of stomach wall 413) which fix the position of the gastric outlet branch 440 in connection with the stomach 412 and the small intestine 410.

An optional feature depicted in connection with the apparatus 420 is the provision of a pass-through passage 480 that is configured to provide a passage through the small intestine 410 in which the passageway 430 is located such that food passing through the small intestine 410 can move from a location upstream of the passageway inlet 432 to a location downstream of the passageway outlet 434 outside of the passageway 430. In other words, the pass-through passage 480 provides an alternate path to pass through the small intestine 410 without having the option of reentering the stomach 412 gastric outlet branch 440.

In one or more embodiments of apparatus including a pass-through passage, the entry of food into the pass-through passage outside of the passageway 430 may be controlled by a pass-through inlet 482 which may, in one or more embodiments, be configured to present a larger or smaller opening within the small intestine 410 to increase or decrease the amount of food allowed to enter into the pass-through passage 480. The pass-through inlet 482 may be, in one or more embodiments, provided in the form of a variable flow restrictor having an opening whose size can be increased or decreased as described in connection with other illustrative embodiments herein. In one or more alternative embodiments, the passageway inlet may, alternatively, have a variable flow restrictor limiting the entry of food into the passageway 430 while the pass-through inlet 482 has a fixed size. In other words, one or both of the passageway inlet 432 and the pass-through inlet 482 may, in one or more embodiments, be configured to change size within the small intestine 410 such that varying amounts of food passing through the small intestine 410 enter the passageway 430 through the passageway inlet 432 or the pass-through passage 480 through the pass-through inlet 482.

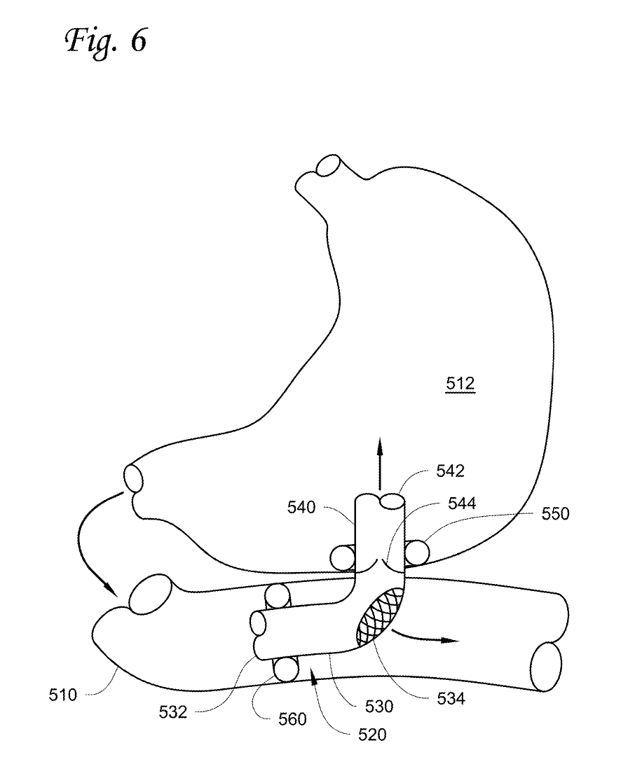

Still another illustrative embodiment of an apparatus 520 configured for gastric recycling is depicted in FIG. 6. The apparatus 520 includes a passageway 530 leading to a gastric outlet branch 540 having a gastric outlet 542 positioned in the stomach 512. The gastric outlet branch 540 includes a gastric outlet fixation apparatus 550 and, also depicted in FIG. 6, is a one-way valve 544 positioned to allow food to enter the gastric outlet branch 540 but restrict the movement of food from the stomach 512 into or through the gastric outlet branch 540.

The apparatus 520 also includes a passageway inlet 532 through which food enters the passageway 530 when traveling downstream through the small intestine 510. A passageway fixation apparatus 560 is provided on the passageway 530 to fix the location of the passageway 530 within the small intestine 510.

An optional feature depicted in connection with the apparatus 520 is a passageway outlet 534 in the form of one or more openings in a wall of the passageway 530. As a result, the apparatus 520 does not have a clearly defined passageway outlet or passageway outlet branch as described in connection with some of the illustrative embodiments above. However, a portion of the food entering the passageway 530 through the passageway inlet 532 will leave the passageway 530 through the one or more openings that form the passageway outlet 534 in the apparatus 530 to enable absorption of nutrients over time.

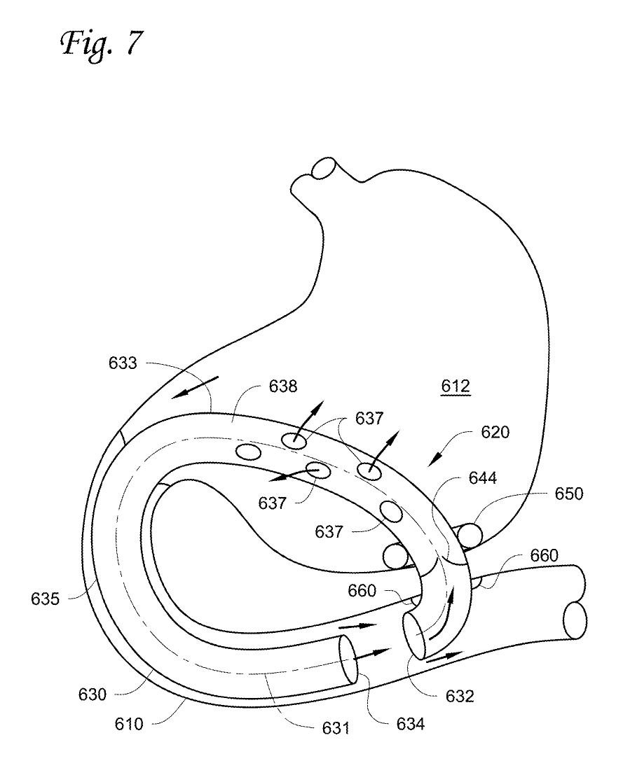

Yet another illustrative embodiment of an apparatus 620 configured to promote gastric recycling is depicted in FIG. 7. The apparatus 620 includes a passageway 630 having a passageway inlet 632, passageway outlet 634 and a one-way valve 644. The apparatus 620 also includes a primary flow axis 631 that extends through the passageway 630 from the passageway inlet 632 to the passageway outlet 634. The passageway 630 includes a gastric portion 633 and a small intestine portion 635. In the illustrative embodiment depicted in FIG. 7, the gastric portion 633 may be described as being located within the stomach 612 and have a distal end and a proximal end positioned to allow recycling of food. The small intestine portion 635 may be described as being located between the gastric portion 633 and the passageway outlet 634. Further, the gastric portion 633 may be described as being located between the passageway inlet 632 and the small intestine portion 635.

The illustrative embodiment of apparatus 620 depicted in FIG. 7 includes, in one or more embodiments, fixation apparatus 660 positioned on an exterior of the passageway 630 within the small intestine 610. In one or more embodiments, the fixation apparatus 660 is configured to fix the position of the passageway 630 within a fistula formed between a stomach 612 and the small intestine 610 such that the gastric portion 633 of the apparatus 620 is located within the stomach 612 and the small intestine portion 635 is located within a small intestine 610.

The fixation apparatus 660 may, in the depicted embodiment, be in the form of sutures, hooks or other mechanical devices configured to locate the passageway inlet 632 at a selected location within a patient. Similar fixation devices may be used in place of the inflatable or expandable fixation devices described in connection with other illustrative embodiments described herein.

In one or more embodiments, the apparatus 620 may also include expandable fixation apparatus 650 located on an exterior of the gastric portion 633 within the stomach 612 of the apparatus 620. Any such expandable fixation apparatus 650 may be constructed according to the descriptions of other expandable fixation apparatus described in connection with other illustrative embodiments described herein.

The gastric portion 633 of the apparatus 620 may, in one or more embodiments, include one or more openings 637 formed in a wall 638 forming the gastric portion 633 of the passageway 630. The one or more opening 637 may be configured to allow food entering the passageway inlet 632 of the passageway 630 in the small intestine 610 to flow out of the one or more openings 637 into the stomach 612. Food moving through the gastric portion 633 of the passageway 630 that does not flow out of the one or more openings 637 into the stomach 612 moves through the small intestine portion 635 of the passageway 630 to the passageway outlet 634.

In one or more embodiments, the passageway inlet 632 of the apparatus 620 may be configured to allow a portion of food moving through the small intestine 610 to pass the passageway inlet 632 without entering the passageway 630. In other words, the apparatus 620 may include a pass-through feature similar to that described above in connection with the apparatus 420 depicted in FIG. 5. As a result, a portion of food passing through the small intestine 610 towards the passageway inlet 632 may pass by the passageway inlet 632 such that it does not enter the passageway 630 and is not, therefore, recycled into the stomach 612.

One difference in the apparatus 620 depicted in FIG. 7 from the other illustrative embodiments of apparatus described herein include the positioning of the passageway outlet 634 upstream from the passageway inlet 632 in the small intestine 610. Although the passageway outlet 634 is depicted in the illustrative embodiment as being relatively close to the passageway inlet 632, in one or more alternative embodiments the passageway outlet 634 may be located much farther upstream from the passageway inlet 632. For example, in one or more embodiments, the passageway outlet may be located at any position downstream from the pyloric valve separating the small intestine portion 635 from the gastric portion 633 of the apparatus 620.

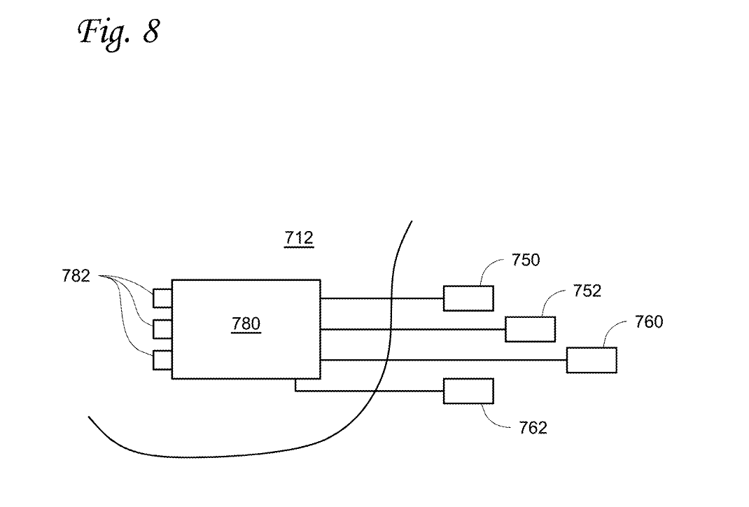

As discussed herein in connection with the illustrative embodiments of gastric recycling apparatus described herein, expandable fixation apparatus may be provided in various locations on the apparatus to maintain its positioning when deployed. In one or more embodiments, where the expandable fixation apparatus are in the form of inflatable balloons or other structures the apparatus may include a port 780 which may provide a location useful to inflate and/or deflate the fixation apparatus. In particular, one illustrative embodiment of a port 780 is depicted in FIG. 8. The port 780 includes connectors 782 which are in fluid communication with one or more of the fixation apparatus 750, 752, 760, 762. In one or more embodiments, the port 780 may be positioned within a stomach 712 of the patient to provide more convenient access to the port 780 so that the fixation apparatus may be selectively inflated and/or deflated.

In one or more embodiments of the gastric recycling apparatus described herein stimulatory electrodes could be placed on the apparatus to potentially spasm or narrow segments of the small intestine periodically to promote movement of food through the apparatus. In one or more embodiments, stimulatory electrodes connected to a power source and controller may be attached to the duodenal muscle layer in the second part of the duodenum (in one or more embodiments, the power source/controller may be located in the abdomen or even lie external to the patient).

In one or more embodiments of the gastric recycling apparatus described herein a spiral conduit may be provided with an inlet and outlet both located within the stomach, the apparatus including a passageway that exits the stomach and wraps around the body and reenters the stomach at another location. In one or more embodiments such an apparatus may delay emptying of the stomach, provide some restraint, and, with stimulatory electrodes, may provide some pacing related benefit as well.

In one or more embodiments, the gastric recycling apparatus described herein may be delivered using a deflectable catheter that has an extrudable needle and wire that can exit from an endoluminal surface and reenter at a more distal location. After re-entry, the deflection mechanism may allow the tip to approximate more proximally, creating a loop or channel over which the gastric recycling apparatus can be placed. In one or more embodiments, clamshell-like structures or equivalents could be built into the gastric recycling apparatus and used to hold it in place along with flow restrictors described herein. One or more alternative embodiments of the apparatus described herein may include dual exit with mating of the two distal ends to create the loop and catheter-based placement of a single flow restrictor distal to the pylorus alone.

In one or more embodiments in which flow restrictors are used in the gastric recycling apparatus described herein, the flow restrictors could be placed all along the conduit or connecting tubes. In one or more embodiments, flow restrictors thus placed could potentially produce phasic movement similar to peristaltic wave, except that the flow restrictors could be operated to promote regurgitation proximally, i.e., in reverse from the normal physiological flow direction. In one or more embodiments, such an apparatus and/or method of operation may reduce the likelihood of clogging. In one or more embodiments, the rate of this created peristaltic wave could be, e.g., varied from an external source or automated based on the amount of ingested food and time of day.

In one or more embodiments that include one or more variable flow restrictors, the flow restrictors could be constructed using, e.g., materials in which an electric charge can produce mechanical conformational changes, including constriction or relaxation (see, e.g. http://www.intrinsicdevices.com/ or http://www.nanosonics.com.au/). Some potentially useful materials for the variable flow restrictors may include, e.g., metals, rubbers, elastic metals, variably elastic rubbers, electrically conformable metalloids, etc.

In one or more embodiments, these materials could be arranged or placed like a ring or spiral to create not only the flow restrictor, but may also provide a variable compression device. In one or more embodiments, the esophageal peristaltic wave and a mechanical- electrical transducer may be used together to create the signal that will cause constriction using the flow restrictor(s) in the pylorus or distal portion of the stomach. In one or more such embodiments, the stasis will gradually develop when food is being ingested.

Although not specifically described, the gastric recycling apparatus described herein may be deployed using any suitable techniques including endoscopes or other devices that may limit the need for surgical procedures. The apparatus may, however, be herein may be deployed by any suitable technique or techniques. Some potentially useful examples of deployment techniques may be described in, e.g., US Patent Application Publication Number 2013/0184635 (Levy et al.).

Disclosure of any patents, patent documents, and publications identified herein are incorporated by reference in their entirety as if each were individually incorporated. To the extent there is a conflict or discrepancy between this document and the disclosure in any such incorporated document, this document will control.

Illustrative embodiments of the obesity treatment apparatus or methods are discussed herein some possible variations have been described. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof. It should also be understood that this invention also may be suitably practiced in the absence of any element not specifically disclosed as necessary herein.

1. An apparatus configured to promote gastric recycling, the apparatus comprising:
a passageway comprising a passageway inlet and a passageway outlet, wherein a primary flow axis extends through the passageway from the passageway inlet to the passageway outlet;
a gastric outlet branch in fluid communication with the passageway at a location between the passageway inlet and the passageway outlet, the gastric outlet branch comprising a gastric outlet configured for placement in a stomach when the passageway is located in a small intestine, wherein the gastric outlet branch defines a gastric outlet flow axis extending through the gastric outlet branch from the passageway to the gastric outlet, and wherein material entering the passageway through the inlet exits the passageway through either the gastric outlet branch or the passageway outlet;
a one-way valve configured to allow movement of material out of the passageway and into the gastric outlet branch and to restrict movement of material from the gastric outlet branch into the passageway; and
gastric fixation apparatus positioned on an exterior of the gastric outlet branch, wherein the gastric fixation apparatus comprises a delivery configuration and an expanded configuration, wherein the gastric fixation apparatus is larger in a radial direction extending outwardly from the gastric outlet flow axis when the gastric fixation apparatus is in the expanded configuration than in the delivery configuration such that movement of the gastric outlet branch through an opening surrounding the exterior of the gastric outlet branch is restricted in at least one direction by the gastric fixation apparatus in the expanded configuration.

2. An apparatus according to claim 1, wherein the passageway inlet is positioned on an inlet branch located upstream from the location at which the gastric outlet branch is in fluid communication with the passageway, and wherein the inlet branch is configured to position the inlet in the small intestine when the passageway and the passageway outlet are located in the small intestine.

3. An apparatus according to claim 2, wherein the apparatus further comprises an inlet branch fixation apparatus positioned on an exterior of the inlet branch, wherein the inlet branch fixation apparatus comprises a delivery configuration and an expanded configuration, wherein the inlet branch fixation apparatus is larger in a radial direction extending outwardly from the primary flow axis when the inlet branch fixation apparatus is in the expanded configuration than in the delivery configuration such that movement of the inlet branch within the small intestine is restricted when the inlet branch fixation apparatus is in the expanded configuration.

4. An apparatus according to claim 1, wherein the passageway inlet is positioned on an inlet branch located upstream from the location at which the gastric outlet branch is in fluid communication with the passageway, wherein the inlet branch is configured position the passageway inlet at or upstream of the pyloric valve when the passageway and the passageway outlet are located in the small intestine, and wherein the inlet branch comprises a sleeve extending through the small intestine to the location at which the gastric outlet branch is in fluid communication with the passageway.

5. An apparatus according to claim 4, wherein the apparatus further comprises an inlet branch fixation apparatus positioned on an exterior of the inlet branch, wherein the inlet branch fixation apparatus comprises a delivery configuration and an expanded configuration, wherein the inlet branch fixation apparatus is larger in a radial direction extending outwardly from the primary flow axis when in the expanded configuration than in the delivery configuration such that movement of the inlet branch downstream from the pyloric valve is restricted when the inlet branch fixation apparatus is in the expanded configuration.

6. An apparatus according to claim 1, wherein the apparatus comprises a pass-through passage configured to provide a passage through a body lumen in which the passageway is located such that material passing through the body lumen can move from a location upstream of the passageway inlet to a location downstream of the passageway outlet outside of the passageway.

7. An apparatus according to claim 6, wherein the pass-through passage comprises a pass-through inlet, and wherein one or both of the passageway inlet and the pass- through inlet are configured to change size within the lumen such that varying amounts of material passing through the body lumen enter the passageway through the passageway inlet or the pass-through passage through the pass-through inlet.

8. An apparatus according to claim 1, wherein the passageway outlet is located in an outlet branch extending away from the location at which the gastric outlet branch is in fluid communication with the passageway such that the passageway outlet is spaced apart from a junction of the outlet branch and the gastric outlet branch.

9. An apparatus according to claim 8, wherein an outlet branch fixation apparatus is positioned on an exterior of the outlet branch, wherein the gastric fixation apparatus comprises a delivery configuration and an expanded configuration, wherein the outlet branch fixation apparatus is larger in a radial direction extending outwardly from the primary flow axis when the outlet branch fixation apparatus is in the expanded configuration than in the delivery configuration such that movement of the outlet branch within a body lumen in which the outlet branch is located is restricted when the outlet branch fixation apparatus is in the expanded configuration.

10. An apparatus according to claim 8, wherein the passageway outlet comprises a variable flow restrictor configured to provide an opening configured to be selectively enlarged or reduced in size to change the flow restriction provided by the variable flow restrictor.

11. An apparatus according to claim 10, wherein the variable flow restrictor comprises a mechanical iris comprising a plurality of movable leaves that are configured to move to change the size of the opening in the variable flow restrictor.

12. An apparatus according to claim 10, wherein the variable flow restrictor comprises an expandable bladder configured to be inflated or deflated to change the size of the opening in the variable flow restrictor.

13. An apparatus according to claim 1, wherein the passageway outlet comprises a plurality of openings in a wall of the passageway.

14. An apparatus configured to promote gastric recycling, the apparatus comprising:
a passageway comprising a passageway inlet and a passageway outlet, wherein a primary flow axis extends through the passageway from the passageway inlet to the passageway outlet, wherein the passageway comprises a gastric portion and a small intestine portion, wherein the gastric portion is located between the passageway inlet and the small intestine portion and wherein the small intestine portion is located between the gastric portion and the passageway outlet;
fixation apparatus positioned on an exterior of the passageway, wherein the fixation apparatus is configured to fix the position of the passageway within a fistula formed between a stomach and a small intestine such that the gastric portion is located within a stomach and the small intestine portion is located within a small intestine; and
one or more openings formed in a wall forming the gastric portion of the passageway, wherein the one or more openings are configured to allow material entering the passageway inlet of the passageway in the small intestine to flow out of the one or more openings, and wherein material moving through the gastric portion of the passageway that does not flow out of the one or more openings moves through the small intestine portion of the passageway to the passageway outlet.

15. An apparatus according to claim 14, wherein the passageway inlet is configured to allow a portion of material moving through a small intestine to pass the inlet without entering the passageway.

16. A method of obesity control, the method comprising:
capturing at least a portion of food passing through a small intestine in a passageway inlet of a passageway located in the small intestine;
delivering at least a portion of food in the passageway into the stomach;
preventing food from passing directly into the passageway from the stomach; and
allowing at least a portion of the food passing through the small intestine to pass through the small intestine downstream of the passageway.

17. A method according to claim 16, wherein delivering at least a portion of food in the passageway into the stomach comprises moving the food out of the passageway through a gastric outlet branch comprising a gastric outlet located in the stomach.

18. A method according to claim 17, wherein a one-way valve located in the gastric outlet branch prevents food from passing directly into the passageway from the stomach.

19. A method according to claim 16, wherein allowing at least a portion of the food passing through the small intestine to pass through the small intestine downstream of the passageway comprises passing the food out of the passageway through a passageway outlet located downstream of the passageway inlet.

20. A method according to claim 16, wherein allowing at least a portion of the food passing through the small intestine to pass through the small intestine downstream of the passageway comprises allowing the food to bypass the passageway inlet such that the food does not enter the passageway.
